(19) United States
(12) Patent Application Publication
(10) Pub. No.: US 2025/0108363 A1
(43) Pub. Date: Apr. 3, 2025

(54) PLASMA-TREATED CATALYST, PRODUCTION METHOD THEREOF AND USE OF THE CATALYST

(71) Applicant: MAX-PLANCK-GESELLSCHAFT ZUR FÖRDERUNG DER WISSENSCHAFTEN E.V., München (DE)

(72) Inventors: Beatriz Roldan Cuenya, Berlin (DE); Weiming Wan, Berlin (DE)

(21) Appl. No.: 18/725,533

(22) PCT Filed: Dec. 22, 2022
(86) PCT No.: PCT/EP2022/087483

§ 371 Date: Jun. 28, 2024

(30) Foreign Application Priority Data
Dec. 30, 2021 (EP) 21218361.0

(51) Int. Cl. B01J 23/89; B01J 35/45; B01J 35/58; B01J 37/03; B01J 37/08; B01J 37/12; B01J 37/34; C07C 1/12

(52) U.S. Cl. CPC B01J 23/8926; B01J 35/45; B01J 35/58; B01J 37/033; B01J 37/08; B01J 37/12; B01J 37/349; C07C 1/12; B01J 2235/30; C07C 2523/89

(57) ABSTRACT

The present invention relates to a catalytically active material, the preparation thereof, and the use of the catalytically active material, e.g. in the catalytic hydrogenation of CO₂ to methanol. The catalytically active material comprising a metal oxide doped with a doping metal, wherein the metal oxide is selected from CeO₂, ZnO, Ga₂O₃, In₂O₃, ZrO₂, Fe₂O₃ and Al₂O₃, the doping metal is selected from Cu, Rd and Au, and the catalytically active material is obtainable by a method comprising a step of non-thermal plasma treatment.

### TECHNICAL FIELD

The present invention relates to a catalytically active material, the preparation thereof, and the use of the catalytically active material.

### BACKGROUND OF THE INVENTION

Sustainable and environmentally friendly production of methanol is important in view of dwindling fossil fuel resources and the threats of climate change. Methanol can be produced from H₂ (green H₂ can be produced by electrolysis through water splitting) and the greenhouse gas CO₂. Today, the synthesis of methanol is carried out from a mixture of H₂, CO and CO₂ over Cu/ZnO/Al₂O₃ catalysts (M. Bukhtiyarova et al., Catal. Letters 147, 416 (2017)). In this method the CO₂ concentration in the starting gas mixture is limited to a maximum of about 10%. Greater concentrations of CO₂ were found to result in reduced methanol selectivity (J. Zhong et al., Chem. Soc. Rev. 49, 1385 (2020)). Accordingly, there is a demand of catalysts which enables hydrogenation of CO₂ to methanol at a high reaction rate and high methanol selectivity.

Further, there is a continued need for active materials that catalyze the reverse water gas shift reaction (CO₂+H₂→CO+H₂O; see e.g. Y. Daza and J. N. Kuhn, RSC Advances 6,49675 (2016)) or the desulfurization of CO₂ feeds or syngas containing CO, H₂ and CO₂, for which traditionally Cu/ZnO catalysts have been used (M. Breysse et al. Catalysis Today 84, 129 (2003); J. W. Bae et al. Int. J. Hydrogen Energy 34, 8733 (2009)).

### Technical Problem

An object of the present invention is to provide a catalytically active material which can be effectively used to catalyze the hydrogenation of CO₂ to methanol and/or other industrially relevant methods such as the reverse water gas shift reaction or the desulfurization of syngas or CO₂ feeds. It is a further object to provide a catalytically active material suitable for the CO₂ hydrogenation to methanol and/or other industrially relevant methods that shows a high activity, high selectivity and/or stability in the corresponding reaction(s). According to one further aspect, it is an object of the present invention to provide a simple and effective method for preparing such catalytically active material that can be suitably used in this method.

### SUMMARY OF THE INVENTION

The present inventors have found that the above problem is solved by the following embodiments (“items”).

### [Item 1]

A catalytically active material comprising a metal oxide doped with a doping metal, wherein
the metal oxide is selected from CeO₂, ZnO, Ga₂O₃, In₂O₃, ZrO₂, Fe₂O₃, and Al₂O₃,
the doping metal is selected from Cu, Pd and Au, and
the catalytically active material is obtainable by a method comprising a step of non-thermal plasma treatment.

### [Item 2]

The catalytically active material according to item 1, wherein
the metal oxide is In₂O₃.

### [Item 3]

The catalytically active material according to item 1 or 2, wherein
the doping metal is Cu.

### [Item 4]

The catalytically active material according to any one of items 1 to 3, wherein
the content of the doping metal with respect to the metal oxide is 0.01 wt % to 5.0 wt %, preferably 0.01 wt % to 3.0 wt %, more preferably 0.05 wt % to 0.3 wt %.

### [Item 5]

The catalytically active material according to any one of items 1 to 4, which is in the form of particles.

### [Item 6]

The catalytically active material according to item 5, wherein
the catalytically active material consists of the metal oxide and the doping metal and optionally negatively charged counterions.

### [Item 7]

The catalytically active material according to item 5 or 6, wherein
the catalytically active material has an average particle diameter of 5 nm to 50 nm, as measured by transmission electron microscopy (TEM) or scanning transmission electron microscopy (STEM).

### [Item 8]

The catalytically active material according to any one of items 1 to 4, which is in the form of film.

### [Item 9]

Use of the catalytically active material according to any one of items 1 to 8 in the catalytic hydrogenation of CO₂ to methanol by reacting a gas mixture consisting of H₂ and CO₂.

### [Item 10]

The use according to item 9, wherein
the gas mixture comprising H₂ and CO₂ is reacted in the presence of the catalytically active material at a pressure of 10 bar to 150 bar, and at reaction temperatures of 100° C. to 400° C., preferably 150° C. to 350° C.

### [Item 11]

The use according to item 9 or 10, wherein
the gas mixture has a H₂/CO₂ molar ratio of 3.0 or more, preferably 3.5 or more.

### [Item 12]

A method for producing the catalytically active material according to any one of items 5 to 7, comprising the steps of
(1) co-precipitating the doping metal and the metal component of the metal oxide as hydroxides by adding an alkaline solution (e.g. Na₂CO₃ solution) to an aqueous solution containing salts of the doping metal and the metal component of the metal oxide,
(2) washing and drying the precipitate,
(3) calcining the dried precipitate in the presence of O₂, and
(4) subjecting the calcined precipitate to a non-thermal plasma treatment to obtain particles of the catalytically active material.

### [Item 13]

The method according to item 12, wherein the non-thermal plasma treatment is carried out at a pressure of 40 mbar or less.

### [Item 14]

A method for producing the catalytically active material according to item 8 comprising the steps of
(1) providing a film comprising the metal of the metal oxide and the doping metal, preferably by a co-deposition method,
(2) annealing the film in the presence of oxygen, and
(3) carrying out a non-thermal plasma treatment.

Where the present description refers to preferred embodiments/features, irrespective of their level of preference, combinations of these preferred embodiments/features shall also be deemed as disclosed, as long as this combination of preferred embodiments/features is technically meaningful.

Herein, the use of the term “comprising” or “including” should be understood as disclosing, as a more restricted embodiment, the term “consisting of” as well, as long as this is technically meaningful.

If preferred upper and lower limits are indicted for certain features, this should be understood as disclosing any combination of the upper and lower limits.

In the following, depending on the context, the term “catalytically active material” may also be replaced by “catalyst”.

### BRIEF DESCRIPTION OF THE DRAWINGS

FIG. 1. Schematic illustrations of the experimental setups used for the plasma treatment of catalytically active materials in the form of films (a) and nanoparticulate powders (b).

FIG. 2. Methanol production rates measured after 1 hour (a) and 24 hours (b) over the pristine and plasma-treated Cu—In₂O₃ and Cu-free In₂O₃ catalysts according to Comparative Examples 1-4, Reference Example 1 and Examples 1-3 (CO₂:H₂:He=1:4:1, 60 bar, 48 mL/min, 280° C.).

FIG. 3. Reaction kinetics measured on the Cu—In₂O₃ catalysts containing 0.1 wt % Cu according to Examples 1, 4 and 5 (CO₂:H₂:He=1:4:1, 60 bar, 48 mL/min, 280° C.).

FIG. 4. Fourier transformed Cu K-edge extended X-ray absorption fine structure (EXAFS) spectra for Cu—In₂O₃ catalysts according to Example 1 (“plasma-treated”) and Comparative Example 1 (“calcined”). Spectra were recorded on each catalyst without any treatment (as-prepared) and after CO₂ hydrogenation reaction at a total pressure of 60 bar in a gas mixture of CO₂+H₂+He (1:4:1) at 280° C. (after reaction). Partial contributions of the Cu—Cu bonds, as obtained from non-linear least-square EXAFS data fitting for the spent catalysts, are shown by the dash lines. Spectra are shifted vertically for clarity.

FIG. 5. Scanning tunneling microscopy images of the In₂O₃ film catalyst (Reference Example 2) and the plasma-treated In₂O₃ film catalyst (Reference Example 3), and the topography profiles (c,d) along the lines shown on the corresponding images (a,b).

FIG. 6. O 1s region in X-ray photoelectron spectra measured for the catalysts according to Reference Example 2 (In₂O₃ pristine), Reference Example 3 (In₂O₃ plasma), Comparative Example 5 (Cu—In₂O₃ pristine) and Example 6 (Cu—In₂O₃ plasma).

### DETAILED DESCRIPTION OF THE INVENTION

### 1. Catalytically Active Material

The catalytically active material of the present invention comprises a metal oxide doped with a doping metal. The metal oxide is selected from CeO₂, ZnO, Ga₂O₃, In₂O₃, ZrO₂, Fe₂O₃ and Al₂O₃. The doping metal is selected from Cu, Pd, and Au. The catalytically active material is obtained by a method comprising a step of non-thermal plasma treatment. This method comprises, for instance, co-precipitation of the metal oxide and the doping metal to obtain a precursor, and subjecting the precursor to calcination.

As “atomically dispersed” we understand the presence of single doping metal atoms (also denoted as “dopant”) in the catalytically active material and optionally also on the surface of the catalytically active material, which can be detected by the usual analytical techniques such as extended X-ray absorption fine structure (EXAFS), infrared spectroscopy (IR), or STEM. For the avoidance of doubt and if not stated otherwise, the term “atom” is used in the description and claims to cover both neutral (oxidation state=0) and charged (ionic, e.g. oxidized) doping metal atoms.

The form of the catalytically active material is not particularly limited. It may be, for instance, in the form of particles (e.g. as powder) or a film.

In order to optimize its usability in industrial processes, the catalytically active material may also be applied to (carried by) inert support materials. If the catalytically active material is present as film, it may be carried by suitable planar support materials.

The present invention is characterized in that the catalytically active material is obtained by a method comprising a step of non-thermal plasma treatment. In accordance with the invention, it is preferred to start with a material where metal atoms of the dopant are incorporated inside the metal oxide (also denoted as “metal oxide matrix”) of the catalytically active material. Among others, the effect of the plasma treatment is considered to be a stabilization of the initial “as prepared” state of the doped metal oxide material, with the doping metal atoms mostly remaining inside the host metal oxide matrix. Without wishing to be bound by theory, if doping metal atoms migrate under reaction conditions from inside the metal oxide matrix to the surface thereof, the doping metal atoms are considered to have a lower mobility on the metal oxide surface due to its larger plasma-enhanced roughness. By contrast, in reference catalysts prepared without plasma treatment, more doping atoms appear to come to the surface (leaving the matrix) under reaction conditions and subsequent sintering takes place. This gives rise in X-ray absorption spectra (XAS) to e.g. metallic Cu—Cu signals corresponding to the formation of relatively large particles, as opposed to having the doping metal atoms dispersed in the In₂O₃ (or the other claimed metal oxides) or making very small clusters.

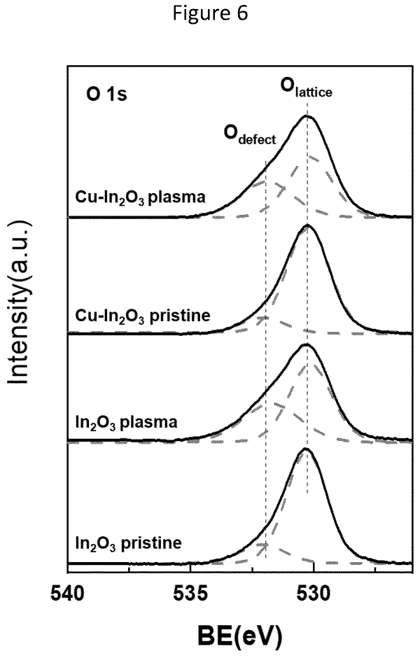

In one embodiment, the catalytically active material of the present invention shows an additional state of 0 atoms characterized by a binding energy of the O 1s core level that is 1.4 eV to 2.5 eV, preferably 1.6 eV to 2.2 eV higher than that of the main signal of the metal oxide as observed by X-ray photoelectron spectroscopy (XPS); see also FIG. 6. The XPS analysis can be carried out according to the method as described in the examples. The additional XPS signal may be observed as a shoulder of the main signal. Without being bound to theory, the additional XPS signal is attributed to oxygen from defect sites, preferably formed on the surface of the metal oxide during the non-thermal plasma treatment. For example, in the case of In₂O₃, the O 1s XPS signal of In₂O₃ appears at 530.2 eV, while that of the oxygen from defect sites appears at 532.0 eV.

In this embodiment, the intensity of the additional signal is preferably at least 30%, more preferably at least 40%, of the intensity of the main peak.

In one embodiment, the non-thermal plasma treatment leads to the formation of metal oxide nanoclusters on the surface of the catalytically active material. The average diameter of the metal oxide nanoclusters may be 4.0 nm or less, preferably 2.5 nm or less, as determined by scanning tunneling microscopy (STM); see further description below. The STM measurements can be carried out as described in the examples.

The catalytically active material of the present invention preferably shows one or more characteristics as described in the above embodiments.

According to the present invention, the catalytically active material comprises a metal oxide doped with a doping metal, wherein the metal oxide is selected from CeO₂, ZnO, Ga₂O₃, In₂O₃, ZrO₂, Fe₂O₃ and Al₂O₃. The metal oxide is preferably selected from ZnO, ZrO₂, Ga₂O₃, In₂O₃, and Al₂O₃, more preferably from ZnO, ZrO₂ and In₂O₃, most preferably In₂O₃. The metal oxide may be used singly, or two or more thereof may be combined.

According to the present invention, the doping metal employed for doping the metal oxide is selected from Cu, Pd and Au. The metal is preferably Cu.

In one preferred embodiment, the “doping” metal atoms are atomically dispersed in the host material (metal oxide matrix).

Preferably, the doping metal is present in the catalytically active material (as prepared) as single atoms (ions) incorporated in the corresponding metal oxide matrix (e.g. Cu inside In₂O₃). It is preferred that the doping metal is present in the as prepared catalytically active material in a positive oxidation state (X), i.e. in oxidized form. In one embodiment, X is 2+ for Cu and Pd, and X is 3+ for Au. It should be noted that the oxidation state may depend on various factors. In the case of Cu—In₂O₃, Cu may e.g. be in the 2+ state, although it substitutes In 3+. To balance the net charge, the process may be accompanied either by creating an O vacancy or by placing a counter ion nearby.

Since the metal oxide is doped with the doping metal, the catalytically active material of the present invention can show excellent catalytic activity in the CO₂ hydrogenation reaction to methanol and/or CO (reverse water-gas-shift), and/or the desulfurization of CO₂ or syngas (CO+CO₂+H₂). Without being bound to theory, it is assumed that the electronic structure of the metal oxide is modified in the presence of the metal dopant, leading to an excellent ability to activate reactants. It is furthermore assumed that the presence of the metal dopant modifies the crystalline structure of the metal oxide, leading to a high surface energy of the metal oxide, which also helps activating reactants.

The combination of the metal oxide and the doping metal is not particularly limited. According to one preferred embodiment, the metal oxide is selected from CeO₂, ZnO, Ga₂O₃, In₂O₃, and Al₂O₃, and the doping metal is selected from Cu, Pd or Au. The combination of In₂O₃ and Cu is more preferable.

The amount of the doping metal with respect to the metal oxide is not particularly limited, and preferably 0.01 wt % to 5.0 wt %, more preferably 0.01 wt % to 3.0 wt %, further preferably 0.01 wt % to 1.0 wt %. The amount of the doping metal with respect to the metal oxide is particularly preferably 0.05 wt % to 0.3 wt %, since the CO₂ hydrogenation activity of the catalytically active material can be further enhanced.

The atmosphere in which the non-thermal plasma treatment is carried out is not particularly limited, and may be one or more selected from N₂, O₂ and Ar, preferably O₂. Preferably, the non-thermal plasma treatment is carried out in the presence of at least O₂.

According to the first embodiment of the present invention, the catalytically active material is present in the form of particles and may e.g. form a powder. The shape of the particles is not particularly limited and may be selected from e.g. ellipsoidal, cubic and spherical shapes.

According to the first embodiment of the present invention, the average particle diameter of the catalytically active material is not limited, and preferably 5 nm to 1 μm, more preferably 5 nm to 500 nm, further preferably 5 nm to 100 nm, particularly preferably 5 nm to 50 nm.

The average particle diameter of the metal oxide substrate can be determined by transmission electron microscopy (TEM) or scanning transmission electron microscopy (STEM) by measuring diameters of 20 arbitrarily selected particles and calculating the average thereof. As “diameter” the longest visible axis of the particle is taken. If the accuracy of the measurement is to be further increased, the measurement can be conducted with 100 arbitrarily selected particles. The detailed method is given in the example section. If several particles form together aggregates, the diameter of the primary particles is taken for the measurement.

According to the first embodiment of the present invention, the catalytically active material preferably consists of the metal oxide and the doping metal. If the doping metal is present in the form of metal ions, (negatively charged) counterions may also be present to balance the net charge. These counterions may e.g. stem from the salts of the doping metal used for the preparation of the catalytically active material.

According to the second embodiment of the present invention, the catalytically active material is in the form of a film.

The catalytically active material as a film may be provided on a base material (substrate). In one embodiment, the base material provides a planar substrate to which the catalytically active material film can be attached, e.g. by deposition of the metal oxide precursor and metal precursor followed by an oxidation step. The base material is not particularly limited, as long as it does not adversely affect the catalytically active material. For example, a base material made of a metal, an alloy or a ceramic may be used. The metal used as the base material may be a single crystal, and example thereof includes a Ru(0001) single crystal. Examples of the alloys that can be used as the base material include, but are not limited to, steel, brass, and the like. Examples of the ceramics that can be used as the base material include, but are not limited to, silica, silicon carbide, and the like. The skilled person is able to select the base material suitable for the reaction conditions to which the catalytically active material (overlayer) is exposed.

According to the second embodiment of the present invention, the thickness of the catalytically active material film is not particularly limited, and preferably 5 nm to 50 nm, more preferably 5 nm to 20 nm. The thickness of the catalytically active material film can be determined by, for example, XPS by measuring the attenuation of an XPS signal of the base material, on which the catalytically active material film is provided. For instance, if the catalytically active material film is provided on a Ru base material, the attenuation of the Ru 3d signal in the XPS spectra can be measured to determine the thickness of the catalytically active material film. Likewise, if a silica base material is used, the attenuation of the Si 2p signal in the XPS spectra can be used. The skilled person is able to appropriately select the signal resulting from the element in the base material and measure the attenuation thereof to determine the thickness of the catalytically active material film.

According to the second embodiment of the present invention, the catalytically active material film may present metal oxide clusters on the film surface, the film and the clusters comprising the same metal oxide. The average diameter of the clusters may be 4.0 nm or less, preferably 2.5 nm or less as determined by scanning tunneling microscopy (STM) by plotting a topography profile along a straight line corresponding to the length of e.g. 10 nm; measuring the distance between two adjacent minima; multiplying the measured value by a correction factor of 0.5 and recording the obtained value as a diameter; repeating the measurement on 20 arbitrarily selected surface areas showing a coverage by metal oxide clusters; and calculating the average value. The detailed method is given in the examples section.

The number of the clusters carried on the on the film surface and normalized to the unit area of the metal oxide substrate (surface cluster density) is not particularly limited. The surface cluster density is preferably at least 0.03 clusters/nm², more preferably at least 0.05 clusters/nm², further preferably at least 0.08 clusters/nm², and most preferably at least 0.10 clusters/nm². The surface cluster density can be determined by STM by counting the number of the metal oxide clusters present within an arbitrarily selected area. The detailed method is given in the examples section.

The catalytically active material of the present invention is particularly suitable for the catalytic hydrogenation of CO₂.

The catalytically active material of the present invention shows an excellent stability and activity in the hydrogenation of CO₂. At least in part, these properties seem to be related to the plasma treatment which modifies the surface structure and/or electronic state of the metal oxide to an extent that, during the catalytic reaction, the formation of large metal clusters (e.g. by aggregation or sintering) is suppressed and the high dispersion of the doping metals is stabilized or enhanced.

The catalytically active material of the present invention can be prepared by the following methods.

### II. Method for Producing the Catalytically Active Material

A method for producing the catalytically active material according to the first embodiment of the present invention may comprise the following steps:
(1) co-precipitating the doping metal and the metal component of the metal oxide as hydroxides by adding an alkaline solution (e.g. Na₂CO₃ solution) to an aqueous solution containing salts of the doping metal and the metal component of the metal oxide,
(2) washing and drying the precipitate,
(3) calcining the dried precipitate in the presence of O₂, and
(4) subjecting the calcined precipitate to a non-thermal plasma treatment to obtain particles of the catalytically active material.

According to step (1), the salt of the doping metal is not particularly limited. Examples include, but are not limited to, nitrate salt, chloride salt, sulfate salt, acetate salt, acetylacetonate salt, tetraamine salt (e.g. tetraaminecopper(II) sulfate, tetraaminepalladium(II) chloride, and the like), tetrachloroaurate(II) salt, and the like. According to step (1), the salt of the metal element of the metal oxide is not particularly limited. Examples include, but are not limited to, nitrate salt, chloride salt, sulfate salt, acetate salt, acetylacetonate salt, tetraamine salt, and the like.

According to step (1), the alkaline solution is not particularly limited, as long as its addition leads to an increase of the pH of the aqueous solution containing the salts of the doping metal and the metal component of the metal oxide. Examples include, but are not limited to, aqueous alkaline solutions of e.g. Na₂CO₃, K₂CO₃, NaOH, KOH, Mg(OH)₂, Ca(OH)₂, CaCO₃, and the like.

As used herein, the expressions “calcining” or “calcination” refers to a thermal treatment of a solid at an elevated temperature in the presence of O₂ under dry conditions. As used herein, the expression “dry” indicates that no additional water is introduced to the atmosphere, in which the calcination is carried out. For example, the calcination in the atmospheric air is to be understood as being carried out under the dry conditions.

The calcination of the precursor for the catalytically active material is preferably carried out at a temperature of 200° C. to 700° C., more preferably 200° C. to 600° C., further preferably 250° C. to 500° C. The calcination is preferably carried out at a concentration of O₂ of 5 vol. % or more, more preferably 10 vol. % or more. For example, air can be used as the source of O₂.

The non-thermal plasma treatment is carried out on the calcined catalytically active material. Prior to ignition of the plasma, the pressure is preferably reduced to 40 mbar or less, more preferably 20 mbar or less. The pressure during the non-thermal plasma treatment is preferably 40 mbar or less, more preferably 20 mbar or less. The atmosphere in which the non-thermal plasma treatment is carried out is not particularly limited, and may be one or more selected from N₂, O₂ and Ar, preferably O₂. Preferably, the non-thermal plasma treatment is carried out in the presence of at least O₂.

The non-thermal plasma treatment may be carried out under a static atmosphere or under a gas flow, the latter being preferred. When the non-thermal plasma treatment is carried out under a gas flow, it is preferred that the calcined precursor for the catalytically active material is blown up (levitated) by the gas flow during the non-thermal plasma treatment.

The source of the non-thermal plasma is not limited, and examples include inductively coupled plasma, capacitively coupled plasma, DC glow discharge, microwave plasma, radio-frequency plasma, cold plasma jet, and dielectric barrier discharge plasma. The source of the non-thermal plasma is preferably microwave plasma or radio-frequency plasma.

When the source of the non-thermal plasma is a microwave plasma, an anode voltage of the plasma source is preferably 0.2 kV to 2 kV, and an emission current is preferably 0.1 μA to 1 μA.

When the source of the non-thermal plasma is the radio-frequency plasma, the power of the radio-frequency plasma generator is preferably 20 W to 300 W.

A duration of the non-thermal plasma treatment can be appropriately adjusted as desired, and is usually 1 min to 300 min, preferably 5 min to 180 min, more preferably 10 min to 120 min, further preferably 30 min to 90 min.

A method for producing the catalytically active material according to the second embodiment of the present invention comprises the steps of
(1) providing a film comprising the metal component of the metal oxide and the doping metal, preferably by a co-deposition method,
(2) annealing the film in the presence of oxygen, and
(3) carrying out a non-thermal plasma treatment.

According to step (1), the method for providing a film comprising the metal oxide doped with the doping metal is not particularly limited, and any method available in the art may be used. Examples include, but are not limited to, co-deposition, sol-gel method, spin coating, and the like. Co-deposition is preferred.

In step (2), the annealing is preferably carried out at a temperature of 200° C. to 400° C., more preferably 250° C. to 350° C., further preferably 280° C. to 320° C. The annealing is preferably conducted in O₂ at pressures of 10⁻⁴ mbar or more.

According to step (3), the non-thermal plasma treatment may be carried out as described above for the method for producing the catalytically active material according to the first embodiment of the present invention.

### III. Catalytic Reaction

The catalytically active material of the present invention can be used in at least one of the following reactions: (i) the hydrogenation of CO₂ to methanol (also referred to as “catalytic hydrogenation”) (ii) the reverse water gas shift reaction (CO₂+H₂→CO+H₂O) or (iii) the desulfurization of syngas (CO+CO₂+H₂) or CO₂ feeds. The CO₂ feeds may, for example, stem from industrial sources like steel mills, trash burning and cement plants.

In one preferred embodiment, the catalytically active material of the present invention is suitably used in the catalytic hydrogenation of CO₂ to methanol. In the catalytic hydrogenation of CO₂ to methanol, a reaction gas mixture comprising H₂ and CO₂ is reacted in the presence of the catalytically active material to produce methanol. The pressure of the reaction gas mixture is not particularly limited and may be for example 10 bar to 300 bar, preferably 20 bar to 100 bar, more preferably 30 bar to 80 bar.

The reaction temperature for the catalytic hydrogenation is not particularly limited and may be 100° C. to 400° C., preferably 150° C. to 350° C.

A composition of the reaction gas mixture is not particularly limited, as long as it comprises CO₂ and H₂. The H₂/CO₂ molar ratio of the reaction gas mixture may be for example 3.0 or more, preferably 3.5 or more, more preferably 4.0 or more. The content of CO₂ in the reaction gas mixture is not particularly limited and may be for example 3 vol % to 25 vol %, preferably 5 vol % to 22 vol %, more preferably 10 vol % to 20 vol %.

The reaction gas mixture may further comprise an inert gas, and examples include N₂, He, Ar, and the like.

### Examples

Herein below, the present invention will be described in more detail with reference to the examples. However, the present invention is not limited to the following Examples.

### Analytical Methods

### Scanning Transmission Electron Microscopy (STEM)

Scanning-transmission electron microscopy (STEM) images of powder catalysts were recorded on the 200 kV JEOL JEM ARM200F probe/image-corrected TEM (JEOL Ltd.).

The average diameter of the catalytically active material was determined by measuring diameters of arbitrarily selected 100 particles and calculating the average thereof. In the event that the STEM image showed a non-spherical shape for the selected particle, the longest axis was taken as diameter.

### X-Ray Photoelectron Spectroscopy (XPS)

XPS spectra were measured with a Phoibos 150 analyzer (SPECS GmbH) using an Al Kα X-ray source (hv=1486.6 eV). Spectral analysis (background subtraction and deconvolution) was performed with the commercial CasaXPS software (Casa Software Ltd; version 2.3.18), and the measured spectrum was subjected to the background subtraction using a Shirley background. O 1s core level spectra were recorded at a pass energy of 20 eV. The skilled person is able to attribute binding energies to chemical species by using the common general knowledge. For instance, tabulated list of binding energies such as NIST X-ray Photoelectron Spectroscopy Database belong to the skilled person's common general knowledge.

### X-Ray Absorption Spectroscopy (XAS)

The Cu K-edge (8979 eV) X-ray absorption spectra (XAS) were measured in the total fluorescence yield mode at ALBA beamline synchrotron radiation facility in Spain using a 4 channel Si drift detector. The measurements were carried out at room temperature in atmospheric air on the catalytically active material in the “as prepared” state and after the CO₂ hydrogenation reaction at 60 bar (total) in CO₂+H₂+He (1:4:1) at 280° C. as described hereinbelow. The extended X-Ray Absorption Fine-Structure (EXAFS) spectra were fitted employing the FEFFIT code and the theoretical backscattering amplitudes and phases, calculated through the FEFF8 code for the reference materials (i.e., Cu, CuO), were used to model the contributions deriving from the Cu—Cu, and Cu—O bonds, respectively.

### Scanning Tunneling Microscopy (STM)

The STM measurements were performed on an In₂O₃(111) film grown on Ru(0001) with a sample bias of 1.8 V and the tunneling current of 0.2 nA.

To determine the average diameter of the metal oxide clusters formed by the plasma treatment on the surface, a topography profile was plotted along a line ca. 10 nm in length. In the topography profile, a distance between two adjacent minima was measured, and the measured value was multiplied by an experimentally derived correction factor of 0.5 to correct for the tip/cluster convolution effects. At least 20 arbitrarily selected metal oxide clusters were measured, and the average corrected value was calculated and used as a “cluster diameter”.

To determine the surface density of metal oxide clusters, i.e. the number of metal oxide clusters normalized to the unit area of the film substrate, the number of metal oxide clusters within an arbitrarily selected 100 nm×100 nm area was counted in the recorded STM images. These measurements were performed in five different sample spots. The mean value was taken as “density of metal oxide clusters”.

### CO₂ Hydrogenation Reaction Over Catalytically Active Material

The reactivity of the catalytically active material in the CO₂ hydrogenation reaction was measured in a tubular packed-bed reactor. 100 mg of a catalytically active material and 100 mg TiO₂ (Alfa Aesar) were physically mixed, and the obtained mixture was loaded into the reactor. TiO₂ is inert for this reaction and was used for dilution. The catalytically active material was heated in 60 bar of He (20 mL/min) at 280° C. for 1 hour. The heated catalytically active material was subsequently cooled down to the room temperature in the He gas flow, and then the gas flow was switched to the reaction mixture consisting of CO₂, H₂, and He (CO₂:H₂:He=1:4:1, 48 mL/min, space velocity GHSV=28800 mL/g_{catalyst}/h).

The pressure was increased to 60 bar and the reduced catalytically active material was heated to 280° C. The gas mixture at the outlet of the reactor was analyzed by gas chromatography (Agilent Technologies 7890B).

### Powder Catalysts

### Comparative Examples 1-3: Preparation of Pristine Cu—In₂O₃ Catalysts

The pristine Cu—In₂O₃ catalysts were prepared following the process as shown below.
1) In(NO₃)₃·xH₂O (Sigma Aldrich) and Cu(NO₃)₂·2.5H₂O (Alfa Aesar) were dissolved in deionized water (50 ml) in a round-bottomed flask (250 ml) to obtain a precursor solution. The amount of In(NO₃)₃·xH₂O used was 2.167 g, and the amount of Cu(NO₃)₂·2.5H₂O was varied as shown below in Table 1.
2) Na₂CO₃ (10.0 g) was dissolved in deionized water (100 ml), and the resulting Na₂CO₃ solution was added dropwise to the precursor solution under stirring at room temperature. The Na₂CO₃ solution was added until the pH reached 9.2. Then the precursor solution became turbid due to the formation of In(OH)₃ and Cu(OH)₂. After aging the slurry for 1 hour, 50 ml deionized water was added.
3) The slurry was centrifuged for 5 min to separate the precipitate, and the precipitate was washed by redispersion in deionized water (50 ml) and subsequent centrifugation for 5 min. This procedure was repeated 5 times to remove the remaining sodium salts.
4) The precipitate was dried in air (70° C., overnight) and then calcined for 3 hours at 300° C. The amounts of Cu(NO₃)₂·2.5H₂O used in the preparation method and the Cu contents of the catalysts are shown in Table 1. The average particle diameters in the catalysts determined by STEM was 15±2 nm.

TABLE 1

| | Cu(NO₃)₂•2.5H₂O | Cu content |
|---|---|---|
| | (mg) | (wt %) |
| Comparative Example 1 | 3.64 | 0.1 |
| Comparative Example 2 | 18.2 | 0.5 |
| Comparative Example 3 | 36.4 | 1.0 |

### Comparative Example 4: Preparation of Pristine In₂O₃ Catalyst

A pristine In₂O₃ catalyst was obtained in the same manner as in Comparative Example 1, except that no Cu(NO₃)₂·2.5H₂O was used. The average particle diameter was 8±1 nm as determined by STEM.

### Examples 1 to 5: Preparation of Plasma-Treated Cu—In₂O₃ Catalysts

Pristine Cu—In₂O₃ catalysts were obtained in the same manner as described in Comparative Example 1 to 3.

Each pristine Cu—In₂O₃ catalyst was then subjected to a plasma treatment in the presence of O₂ following the process as shown below, thereby obtaining plasma-treated Cu—In₂O₃ catalysts.

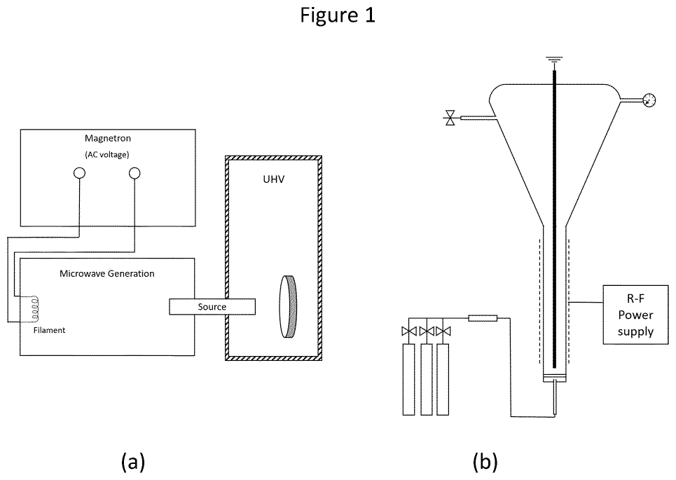

The plasma treatment was carried out in a setup which consisted of a glass tube with frits, a funnel shape glassware, a mechanical pump, a radiofrequency plasma generator and a high voltage power supply. The glass tube was wrapped with a Cu mesh which was connected to a high voltage power supply. The frits could hold the calcined powder while allowing the gas (O₂ was used herein) to blow from the bottom. The funnel-shaped glassware connected to the top of the tube and the other 3 outlets of the funnel-shaped glassware were connected to a pressure meter, mechanical pump and a tungsten bar for grounding. The tungsten bar extended to the bottom of the glass tube to achieve a uniformly distributed plasma. A schematic illustration of the setup for the plasma treatment is provided in FIG. 1b.

The plasma was generated by a 20-60 kHz high voltage power supply (PVM500). The peak voltage was measured by an oscilloscope with a high voltage probe and the power of the plasma was the product of the square root of the mean square (RMS) voltage and RMS current, which were measured by a multimeter. The power and frequency outputs of the powder supply were adjusted until a plasma with a desirable power was formed.

The conditions of the plasma treatment were as follows:
1) The pristine Cu—In₂O₃ powder catalyst was introduced into the glass tube (amount between 100-300 mg) and the tube was evacuated to below 20 mPa.
2) Oxygen flowed from the bottom of the tube with a flow rate 20 mL/min O₂ set by a mass flow controller (MFC).
3) The powder sample started to levitate in a turbulent flow.
4) The plasma was ignited. The power output and frequency of the power supply was adjusted to achieve the target power as shown in Table 2.
5) The plasma treatment took 1 hour.

The pristine Cu—In₂O₃ catalysts used for the plasma treatment, the target power of the plasma treatment and the Cu contents of the catalysts are shown in Table 2. The average particle diameters of the catalysts was 16±2 nm as determined by STEM.

TABLE 2

| | Pristine Cu—In₂O₃ | Target | Cu content |
|---|---|---|---|
| | catalyst used | power (W) | (wt %) |
| Example 1 | Comparative Example 1 | 200 | 0.1 |
| Example 2 | Comparative Example 2 | 200 | 0.5 |
| Example 3 | Comparative Example 3 | 200 | 1.0 |
| Example 4 | Comparative Example 1 | 100 | 0.1 |
| Example 5 | Comparative Example 1 | 170 | 0.1 |

### Reference Example 1: Preparation of Plasma-Treated In₂O₃ Catalyst

A plasma-treated In₂O₃ catalyst was prepared by subjecting the pristine In₂O₃ catalyst of Comparative Example 4 to a plasma treatment in the same manner as described in Examples 1 to 3. The average particle diameter was 10 nm as determined by STEM.

### Evaluation of the Results

As used herein, the “pristine” catalysts will be also referred to as “calcined” catalysts. The prepared Cu—In₂O₃ catalysts had an average diameter of 16±2 nm as measured by STEM. The general morphology of the catalysts was not affected by the plasma treatment under the conditions used.

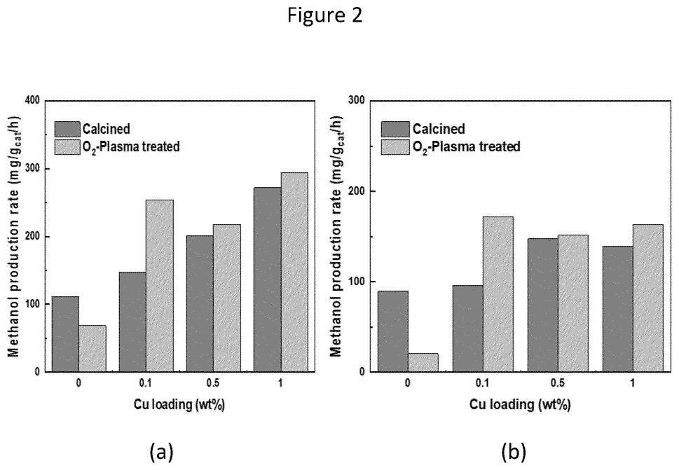

The CO₂ hydrogenation activity of the prepared catalytically active materials were measured as described above. FIG. 2a shows the methanol production rates measured for the (Cu—)In₂O₃ catalysts of Comparative Examples 1-4, Reference Example 1 and Examples 1-3 after 1 hour of reaching the reaction temperature of 280° C. As seen therefrom, for the pristine (Cu—)In₂O₃ catalysts of Comparative Examples 1-4, a higher methanol production rate was observed with the higher Cu loading. The plasma-treated Cu-free In₂O₃ catalyst of Reference Example 1 showed a lower methanol production rate than the pristine In₂O₃ catalyst of Comparative Example 4. To the contrary, the plasma-treated Cu—In₂O₃ catalysts of Examples 1-3 showed a higher methanol production rate than the pristine Cu—In₂O₃ catalyst of Comparative Examples 1-3 at the same Cu loading. The enhancement of the methanol production rate by O₂-plasma treatment was especially strong at the lower Cu loading of 0.1 wt %.

FIG. 2b shows the methanol production rates measured for the (Cu—)In₂O₃ catalysts of Comparative Examples 1-4, Reference Example 1 and Examples 1-3 after 24 hours reaching the reaction temperature of 280° C. As seen therefrom, the pristine Cu—In₂O₃ catalysts of Comparative Examples 1-3 showed a higher methanol production rate than the pristine In₂O₃ catalyst of Comparative Example 4. Among the pristine (Cu—)In₂O₃ catalysts of Comparative Examples 1-4, the highest methanol production rate was observed at the Cu loading of 0.5 wt %. The plasma-treated In₂O₃ catalyst of Reference Example 1 showed a lower methanol production rate than the pristine In₂O₃ catalyst of Comparative Example 4. To the contrary, the plasma-treated Cu—In₂O₃ catalysts of Examples 1-3 showed a higher methanol production rate than the pristine Cu—In₂O₃ catalyst of Comparative Examples 1-3 at the same Cu loading. The enhancement of the methanol production rate by O₂-plasma treatment was especially strong at the Cu loading of 0.1 wt %.

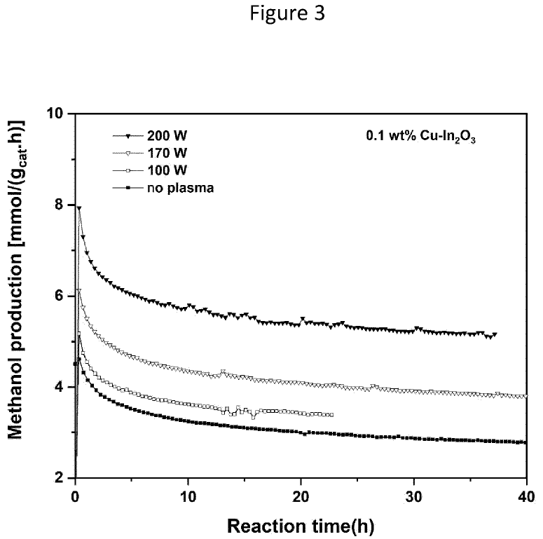

FIG. 3 shows reaction kinetics measured on the Cu—In₂O₃ catalysts containing 0.1 wt % Cu according to Examples 1, 4 and 5, wherein the target power of the plasma treatment was varied from 100 W to 200 W. The reaction rate gradually increased with increasing the power of the plasma employed from 100 to 200 W.

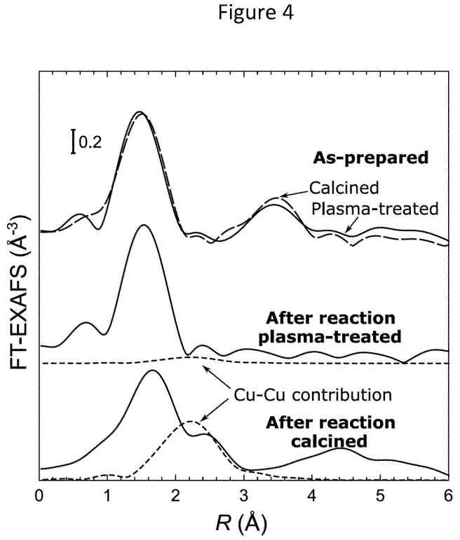

FIG. 4 shows Fourier-transformed Cu K-edge EXAFS spectra for the Cu—In₂O₃ catalysts prepared according to Example 1 (plasma-treated) and Comparative Example 1 (calcined). Measurements were carried out on each catalyst “as-prepared” and after CO₂ hydrogenation reaction performed at 60 bar (total) in CO₂+H₂+He (1:4:1) at 280° C. as described in the foregoing (“after reaction”). The spectra measured for the “as-prepared” Cu—In₂O₃ catalysts before and after the O₂-plasma pre-treatment revealed a similar structure, which is dominated by the Cu—O distance in the first coordination shell. Another contribution to the Cu K-edge EXAFS spectra for as-prepared samples was observed at ca. 3.5 Å, with the position similar to that corresponding to the In—In contribution in In₂O₃. We therefore assign the presence of this feature to the Cu substitution of an In atom in the In₂O₃ lattice. After the reaction, the calcined Cu—In₂O₃ catalyst showed a significant number of Cu—Cu bonds, indicating the agglomeration of Cu atoms. On the other hand, the plasma treated Cu—In₂O₃ sample retained the Cu—O bonds. The Cu—O and Cu—Cu coordination numbers, obtained from the fit of the experimental Cu K-edge EXAFS data, are shown in Table 3 below. Uncertainties of the last digit are given in parentheses. Spectra for all samples were fitted simultaneously, constraining the bond length and disorder factors for Cu—O and Cu—Cu bonds to be the same for all spectra. The obtained length of Cu—O bond was 1.944±0.006 Å, the obtained length of the Cu—Cu bond is 2.542±0.006 Å, corresponding disorder factors were 0.005±0.006 Å² and 0.007±0.002 Å², respectively. Corrections to photoelectron reference energy (ΔE₀) obtained in the fit, as well as the valued of R-factors of the fit are also shown in Table 3.

TABLE 3

| | N_{Cu—O} | N_{Cu—Cu} |
|---|---|---|
| Comparative Example 1 (as-prepared) | 3.3(3) | 0.3(3) |
| Comparative Example 1 (after reaction) | 2.2(2) | 2.2(4) |
| Example 1 (as-prepared) | 3.3(3) | 0.1(4) |
| Example 1 (after reaction) | 3.1(2) | 0.2(3) |

This result shows the difference between the plasma-treated and calcined Cu—In₂O₃ samples after reaction under identical conditions and confirms that the plasma treatment can suppress the agglomeration of Cu atoms that might segregate to the surface of the doped metal oxide during catalytic operation.

### Film Catalysts

### Reference Example 2: Preparation of In₂O₃ Film Catalyst

The experiments were performed in an ultrahigh vacuum (UHV) chamber equipped with low energy electron diffraction (LEED), X-ray photoelectron spectroscopy (XPS), and scanning tunneling microscopy (STM), all from SPECS GmbH. The well-ordered In₂O₃(111) film was grown on a Ru(0001) single crystal as follows. The Ru(0001) single crystal (9 mm in diameter, 1.5 mm in thickness, from MaTeck GmbH) was mounted onto a stainless-steel sample holder having a hole of 9 mm in diameter for heating the sample from the backside using an electron beam from a W filament. A type K thermocouple was spot-welded to the edge of the crystal. The surface of the Ru(0001) was oxidized in 10⁻⁶ mbar of O₂ at 1000 K and cooled down to room temperature in oxygen. Indium was vapor-deposited onto the oxidized Ru(0001) surface using an electron beam assisted evaporator (Focus EMT3) from a Mo crucible filled with In (99.9%, Sigma Aldrich) in 10⁻⁶ mbar of O₂ at 90 K in amounts equivalent to form 4-5 monolayers (MLs) of In₂O₃(111). Subsequently, the temperature of the Ru(0001) was increased at a rate of 1 K/s, and kept at 673 K during the deposition of further In₂O₃ layers. The sample was then oxidized at 1000 K in 10⁻⁶ mbar of O₂ to improve the film crystallinity. The thickness of the prepared In₂O₃(111) film was about 5 nm. The prepared sample is denoted as “In₂O₃(111)”.

### Comparative Example 5: Preparation of Pristine Cu—In₂O₃ Film Catalyst

Cu and In were co-deposited onto the In₂O₃(111) film using two electron beam-assisted evaporators (Omicron EMT3) from Mo crucibles each filled with Cu and In, respectively. Approximately 0.1 ML Cu and 0.9 ML of In was deposited on the In₂O₃(111) surface. As used herein, 1.0 ML corresponds to one atom per In₂O₃(111) surface unit cell, i.e. 5.6×10¹³ atoms/cm². The flux of Cu and In were 0.02 ML/min and 0.1 ML/min, respectively. The sample was then oxidized at 573 K in 10⁻⁵ mbar of O₂. The obtained sample is denoted as “pristine-Cu—In₂O₃”.

### Example 6: Preparation of Plasma-Treated Cu—In₂O₃ Film Catalyst

A pristine Cu—In₂O₃ film catalyst was prepared in the same manner as in Comparative Example 6. The pristine Cu—In₂O₃ film catalyst was then subjected to the nonthermal plasma-treatment in the presence of O₂ following the process as shown below, thereby obtaining the plasma-treated Cu—In₂O₃ film catalyst, denoted as “plasma-Cu—In₂O₃”.

The oxygen plasma treatment of the pristine-Cu—In₂O₃ film catalyst was carried out with a microwave plasma generator with a commercial plasma source (OSPrey, from Oxford Scientific, FIG. 1a) operated in 7×10⁻⁶ mbar of O₂ at an anode voltage of 1 kV and an emission current of 0.5 μA. The duration of the plasma treatment was 1 hour.

### Reference Example 3: Preparation of Plasma-Treated In₂O₃ Film Catalyst

The oxygen plasma treatment of the In₂O₃ film catalyst of Reference Example 2 was carried out in the same manner as described in Example 6.

### Evaluation of the Results

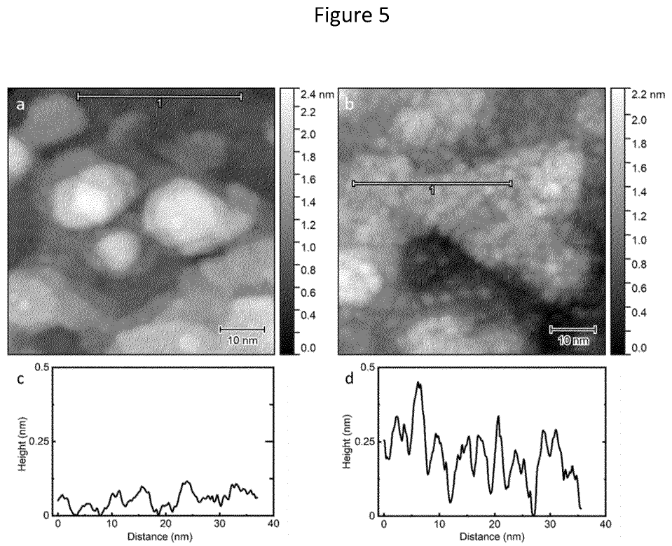

The surface morphologies of the In₂O₃ film catalysts of Reference Examples 2 and 3 were investigated by using STM. The STM image of the plasma-treated In₂O₃ film catalyst according to Reference Example 3 (FIG. 5b) revealed the formation of In₂O₃ clusters with a broad size distribution that ranges between 1 and 4 nm in diameter randomly distributed on the surface of the plasma-treated In₂O₃ film catalyst. The number of the In₂O₃ clusters normalized to the unit area of the STM image was determined to be 0.08 clusters/nm². Such In₂O₃ clusters were not observed in the not plasma-treated In₂O₃(111) film catalyst of Reference Example 2 (see FIG. 5a).

The XPS O 1s spectra of the (Cu—)In₂O₃ catalysts revealed that, after the plasma treatment, a shoulder appeared (FIG. 6). This shoulder is assigned to the oxygen atoms in defect sites (denoted as “O_{defect}”). This result is consistent with the STM image that more clusters were created by plasma treatment, which provide a stronger shoulder in the O 1s spectra.

1. A catalytically active material comprising a metal oxide doped with a doping metal, wherein the metal oxide is selected from CeO₂, ZnO, Ga₂O₃, In₂O₃, ZrO₂, Fe₂O₃, and Al₂O₃,
the doping metal is selected from Cu, Pd and Au, and
the catalytically active material is obtainable by a method comprising a step of non-thermal plasma treatment.

2. The catalytically active material according to claim 1, wherein the metal oxide is In₂O₃.

3. The catalytically active material according to claim 1, wherein the doping metal is Cu.

4. The catalytically active material according to claim 1, wherein a content of the doping metal with respect to the metal oxide is 0.01 wt % to 5.0 wt %.

5. The catalytically active material according to claim 1, wherein the catalytically active material is in the form of particles.

6. The catalytically active material according to claim 5, wherein the catalytically active material consists of the metal oxide and the doping metal and optionally negatively charged counterions.

7. The catalytically active material according to claim 5, wherein the catalytically active material has an average particle diameter of 5 nm to 50 nm, as measured by transmission electron microscopy or scanning transmission electron microscopy.

8. The catalytically active material according to claim 1, wherein the catalytically active material is in the form of film.

9. A method for a catalytic hydrogenation of CO₂ to methanol, comprising using the catalytically active material according to claim 1 by reacting a gas mixture consisting of H₂ and CO₂.

10. The method according to claim 9, wherein the gas mixture comprising H₂ and CO₂ is reacted in the presence of the catalytically active material at a pressure of 10 bar to 150 bar, and at reaction temperatures of 100° C. to 400° C.

11. The method according to claim 9, wherein the gas mixture has a H₂/CO₂ molar ratio of 3.0 or more.

12. A method for producing the catalytically active material according to claim 5, comprising the steps of
(1) co-precipitating the doping metal and the metal component of the metal oxide as hydroxides by adding an alkaline solution to an aqueous solution containing salts of the doping metal and the metal component of the metal oxide,
(2) washing and drying the precipitate,
(3) calcining the dried precipitate in the presence of O₂, and
(4) subjecting the calcined precipitate to a non-thermal plasma treatment to obtain particles of the catalytically active material.

13. The method according to claim 12, wherein the non-thermal plasma treatment is carried out at a pressure of 40 mbar or less.

14. A method for producing the catalytically active material according to claim 8 comprising the steps of
(1) providing a film comprising the metal of the metal oxide and the doping metal by a co-deposition method,
(2) annealing the film in the presence of oxygen, and
(3) carrying out a non-thermal plasma treatment.

15. The catalytically active material according to claim 4, wherein a content of the doping metal with respect to the metal oxide is 0.01 wt % to 3.0 wt % or 0.05 wt % to 0.3 wt %.

16. The method according to claim 10, wherein the gas mixture comprising H₂ and CO₂ is reacted in the presence of the catalytically active material at a pressure of 10 bar to 150 bar, and at reaction temperatures of 150° C. to 350° C.

17. The method according to claim 11, wherein the gas mixture has a H₂/CO₂ molar ratio of 3.5 or more.

18. The catalytically active material according to claim 2, wherein the doping metal is Cu.

19. The catalytically active material according to claim 6, wherein the catalytically active material has an average particle diameter of 5 nm to 50 nm, as measured by transmission electron microscopy or scanning transmission electron microscopy.

20. The method according to claim 12, wherein the alkaline solution is Na₂CO₃ solution.
